(19) United States
(12) Patent Application Publication
(10) Pub. No.: US 2022/0079899 A1
(43) Pub. Date: Mar. 17, 2022

(54) COMPOSITIONS AND METHODS FOR TREATING OCULAR CONDITIONS

(71) Applicant: The Regents of the University of California, Oakland, CA (US)

(72) Inventor: Arjun Deb, Los Angeles, CA (US)

(21) Appl. No.: 17/421,453

(22) PCT Filed: Jan. 13, 2020
(86) PCT No.: PCT/US2020/013345

§ 371 Date: Jul. 8, 2021

Related U.S. Application Data
(60) Provisional application No. 62/792,206, filed on Jan. 14, 2019.

(51) Int. Cl. A61K 31/192; A61K 31/65; A61K 31/194; A61K 31/165; A61K 31/4402; A61K 31/375; A61K 31/4166; A61K 31/5415; A61K 31/10; A61K 31/138; A61K 31/353; A61K 31/198; A61K 31/221; A61K 31/52; A61K 31/546; A61K 31/137; A61K 31/4745; A61K 31/05; A61K 9/00

(52) U.S. Cl. CPC A61K 31/192; A61K 9/0048; A61K 31/194; A61K 31/165; A61K 31/4402; A61K 31/375; A61K 31/4166; A61K 31/5415; A61K 31/10; A61K 31/138; A61K 31/353; A61K 31/198; A61K 31/221; A61K 31/52; A61K 31/546; A61K 31/137; A61K 31/4745; A61K 31/05; A61K 31/65

(57) ABSTRACT

Disclosed herein are methods for treating an ocular pathology in a subject, comprising administering to the subject an ENPP1 inhibitor. Also disclosed are methods of inhibiting ATP hydrolysis in ocular tissue, the method comprising contacting the ocular tissue with an ENPP1 inhibitor. Also provided herein are ectonucleotide pyrophosphatase/phosphodiesterase-1 (ENPP1) inhibitors and compositions comprising the same.

### RELATED APPLICATIONS

This application claims the benefit of U.S. Provisional Application No. 62/792,206, filed Jan. 14, 2019, the contents of which are fully incorporated by reference herein.

### STATEMENT REGARDING FEDERALLY SPONSORED RESEARCH

This invention was made with government support under Grant Number W81XWH-17-1-0464, awarded by the Army Medical Research and Materiel Command and Grant Numbers HL129178, HL137241, awarded by the National Institutes of Health. The government has certain rights in the invention.

### BACKGROUND

Mammalian tissues calcify with age and injury. Analogous to bone formation, osteogenic cells are thought to be recruited to the affected tissue and induce mineralization. Calcification of soft tissues is a cell-mediated process that resembles bone formation in the skeletal system with calcification of the extracellular matrix by cells capable of mineralization. Pathological mineralization of soft tissues, or ectopic calcification, commonly occurs with tissue injury and degeneration and in common diseases such as diabetes and chronic kidney disease. In the orphan disease of pseudoxanthoma elasticum (PXE), ectopic calcification can occur in several types of soft tissues. In the eyes, ectopic calcification of the Bruch's membrane is characteristic and can lead to retinal damage and blindness. In the cardiovascular system, the later stages of PXE often feature angina and myocardial infarction. PXE can occur in young individuals as well as in the elderly.

PXE is caused by deficiency of ABCC6 and is characterized by increased levels of the enzyme ectonucleotide pyrophosphatase/phosphodiesterase-1 (ENPP1), which breaks down ATP to AMP and pyrophosphate (PPi). The balance of extracellular phosphate (Pi) and pyrophosphate critically regulates calcification of the extracellular matrix. Pyrophosphate generated at the cell surface by ENPP1 promotes mineralization by serving as a substrate for tissue non-specific alkaline phosphatase that hydrolyzes pyrophosphate to generate inorganic phosphate. Thus, inhibition of ENPP1 reduces the amount of PPi formed and subsequent calcification.

New treatments to retard calcification in soft tissues, blood vessels or valves, or to inhibit pathological calcification of tissues are needed.

### SUMMARY

In certain aspects, the present disclosure provides methods of treating an ocular pathology characterized by ectopic calcification in a subject, comprising administering to the subject an ENPP1 inhibitor. In certain embodiments, the ocular pathology is pseudoxanthoma elasticum (PXE), sclero-choroidal calcification or choroidocalcinosis.

In certain aspects, the present disclosure provides pharmaceutical preparations, e.g., suitable for use in a human patient, in the treatment of an ocular pathology characterized by ectopic calcification, comprising an effective amount of an ENPP1 Inhibitor, such as any of the compounds described herein, and one or more pharmaceutically acceptable excipients. Such pharmaceutical preparations may be for use in treating or preventing a condition or disease as described herein.

In certain aspects, the present disclosure provides methods of inhibiting ATP hydrolysis in ocular tissue, the methods comprising contacting the ocular tissue with an ENPP1 inhibitor, such as one of the compounds disclosed herein.

### BRIEF DESCRIPTION OF THE DRAWINGS

FIGS. 1A and 1B show calcification in injured hearts in a mouse model of PXE treated with vehicle (A) or ARL67156 (B).

### DETAILED DESCRIPTION

It has been reported that ENPP1 inhibitors can be used to prevent ectopic calcification (Cell Stem Cell (2017) 20:1-15). Primary and secondary screening of compounds has now identified small molecule inhibitors of ENPP1, and a monoclonal antibody has now been generated targeting the extracellular catalytic domain of ENPP1. As inhibition of ENPP1 can decrease ectopic calcification, ENPP1 inhibitors represent a novel therapeutic strategy for ocular pathologies such as PXE.

The disclosed methods provide inhibitors of ENPP1, which substantially inhibit ectopic calcification.

Several ENPP1 inhibitors are known in the art. For example, rosmarinic acid (also known as SYL-001) has the following structure:

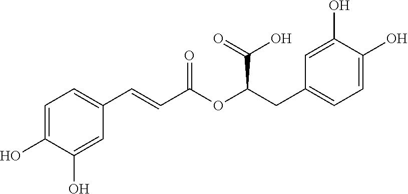 [SMILES: O=C(/C=C/c1ccc(O)c(O)c1)O[C@H](Cc1ccc(O)c(O)c1)C(=O)O]

and is known for its activity as an anti-oxidant and GABA transaminase inhibitor. (See, Sassi, et al. J. Clin. Invest. 2014 124:5385-5397.) Another ENPP1 inhibitor is ARL67156, which has the following structure:

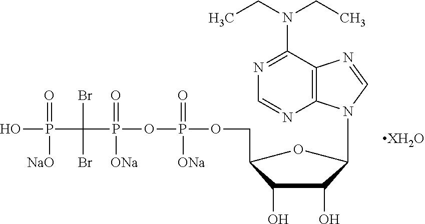

Its ENPP1 inhibitory activity has been described by Cote et al. (Eur. J. Pharmacol. 2012 689:139-146) and Levesque et al. (Br. J. Pharmacol. 2007 152:141-150). A third compound with ENPP1 inhibitory activity is a bisphosphonate known as etidronic acid:

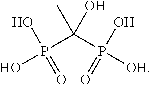

Although primarily used for their anti-resorptive effect on bone, first generation bisphosphonates such as etidronic acid can bind to calcium hydroxyapatite in sites of active bone remodeling and, as they are not hydrolyzable, prevent further bone mineralization. It is also an antagonist to vascular mineralization.

In certain aspects, the present disclosure provides methods of treating an ocular pathology characterized by ectopic calcification in a subject, comprising administering to the subject an ENPP1 inhibitor. In certain embodiments, the ocular pathology is pseudoxanthoma elasticum (PXE), sclero-choroidal calcification, or choroidocalcinosis. In certain preferred embodiments, the ocular pathology is PXE. In other preferred embodiments, the ocular pathology is sclero-choroidal calcification. In further preferred embodiments, the ocular pathology is choroidocalcinosis.

In certain embodiments, the methods comprise administering the ENPP1 inhibitor ocularly. In certain embodiments, the ocular administration is topical (e.g., as eyedrops). In other embodiments, the ocular administration is by intraocular injection. In certain embodiments, the ocular administration is via implantation of a device comprising the ENPP1 inhibitor to an eye of the subject.

In certain embodiments, the subject is an adult, e.g., an elderly subject. In other embodiments, the subject is a pediatric subject.

In certain embodiments, the ENPP1 inhibitor is selected from

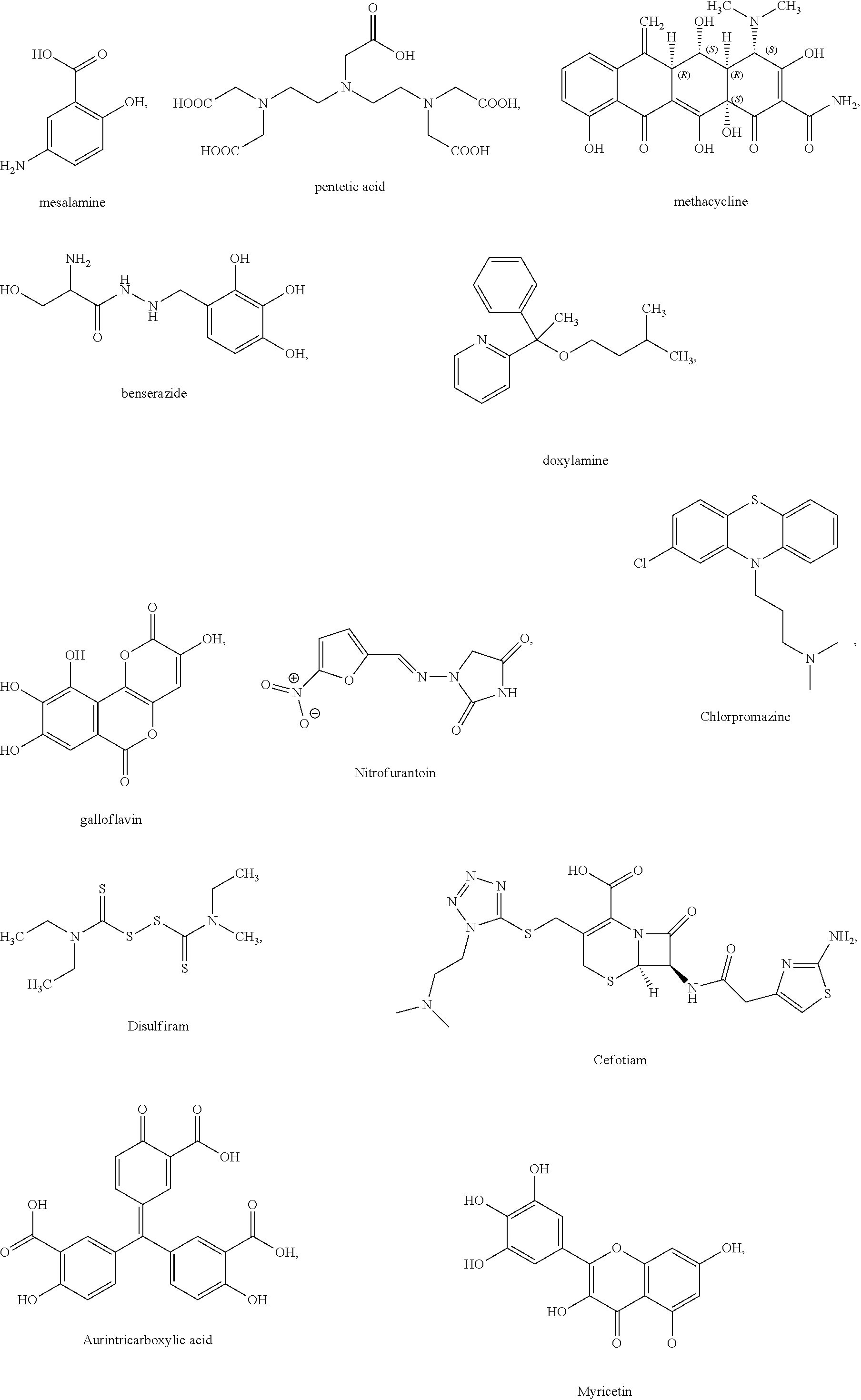

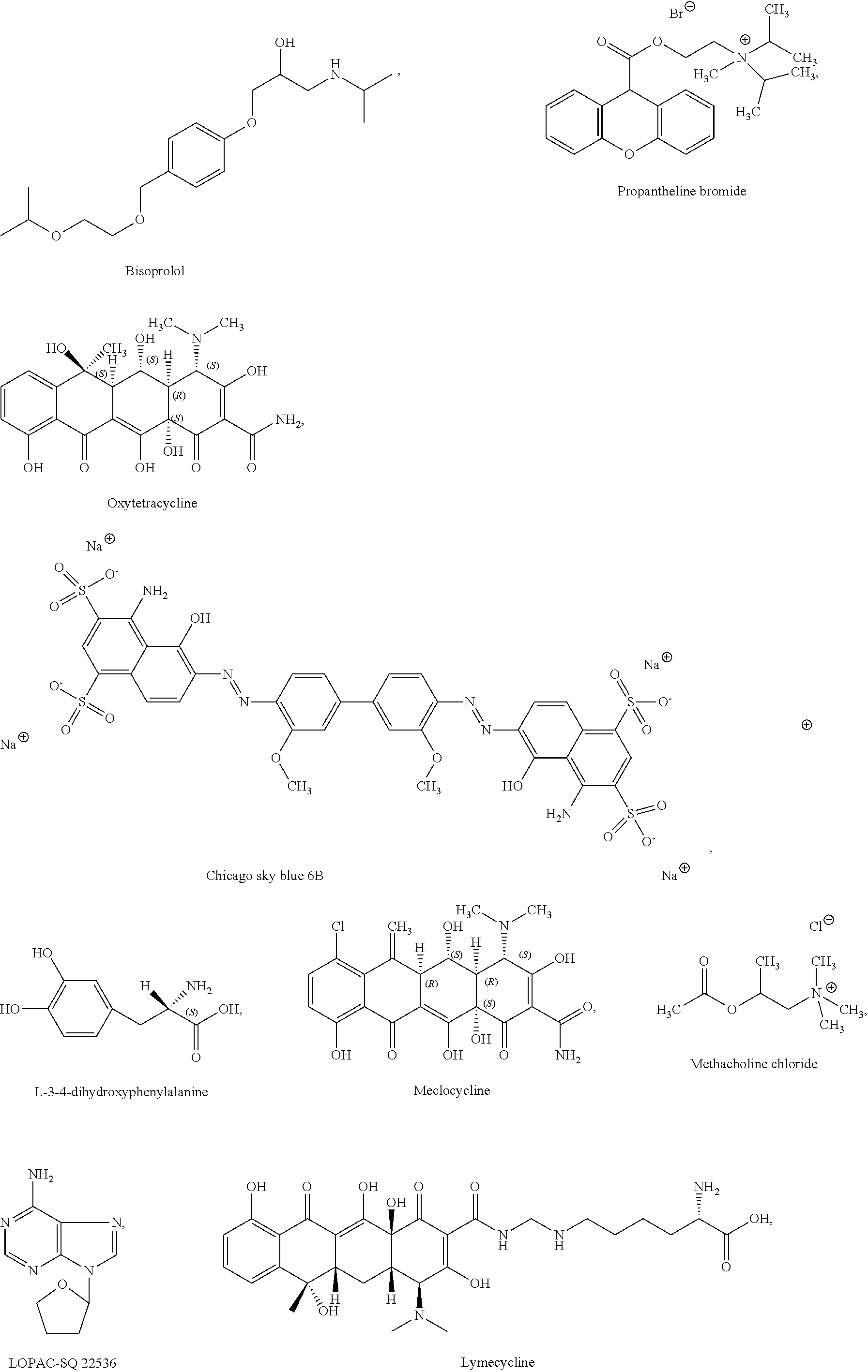

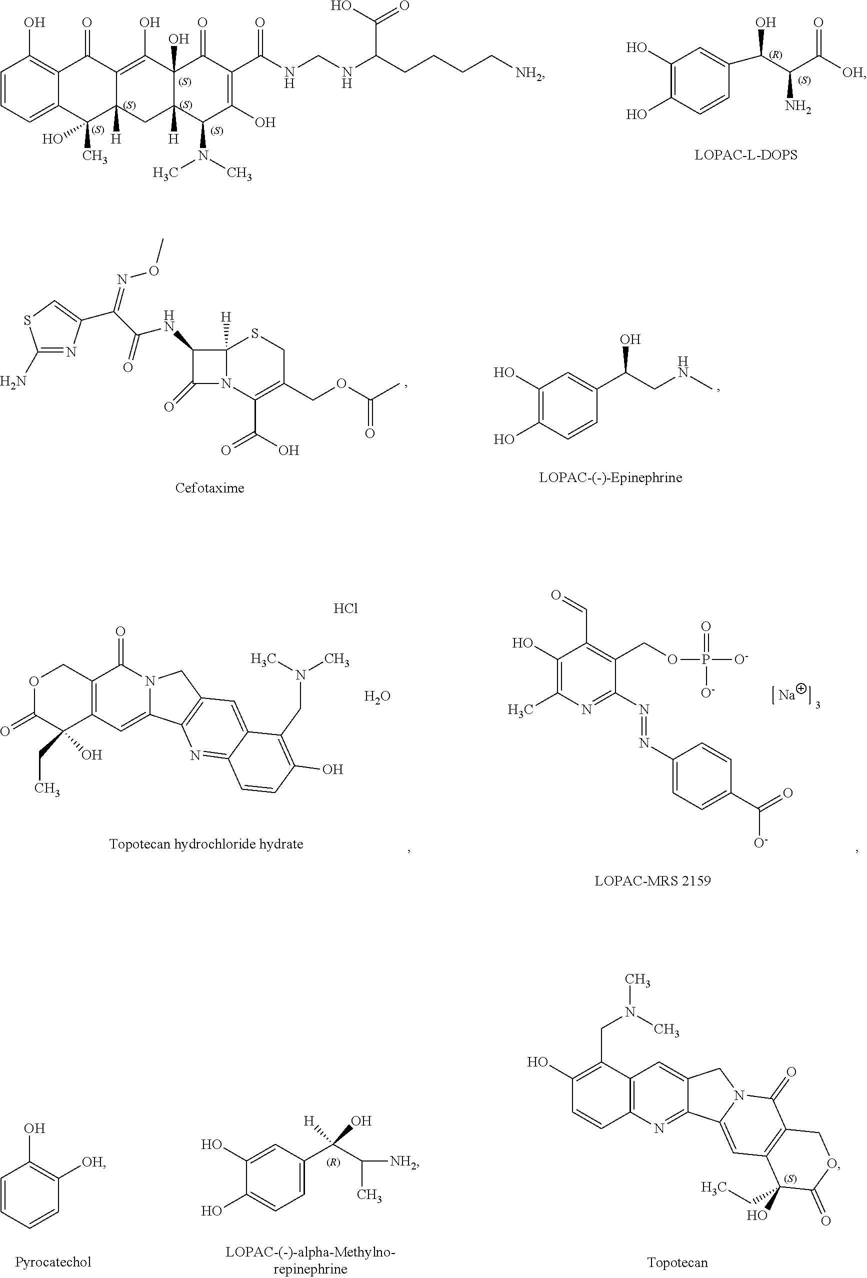

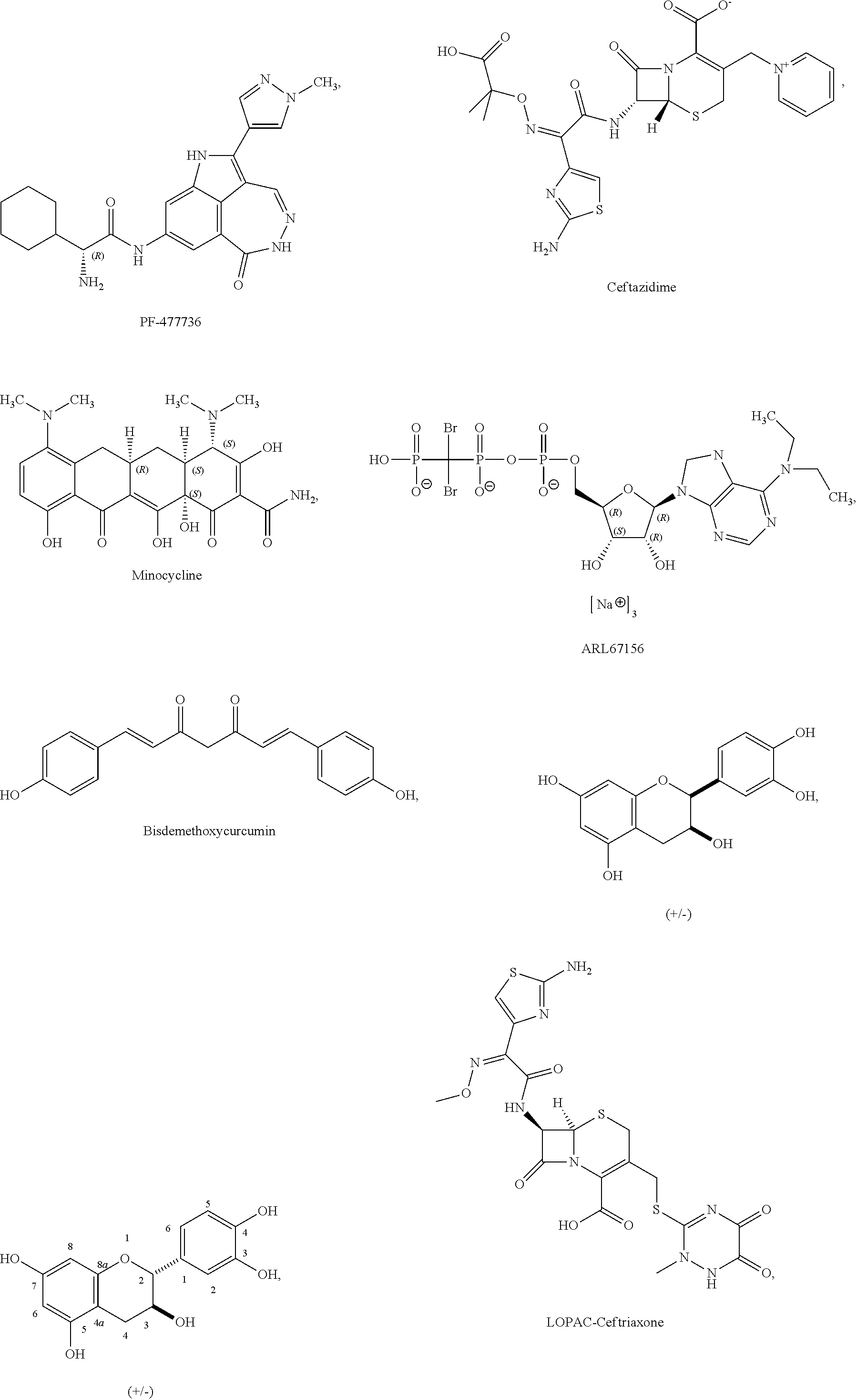

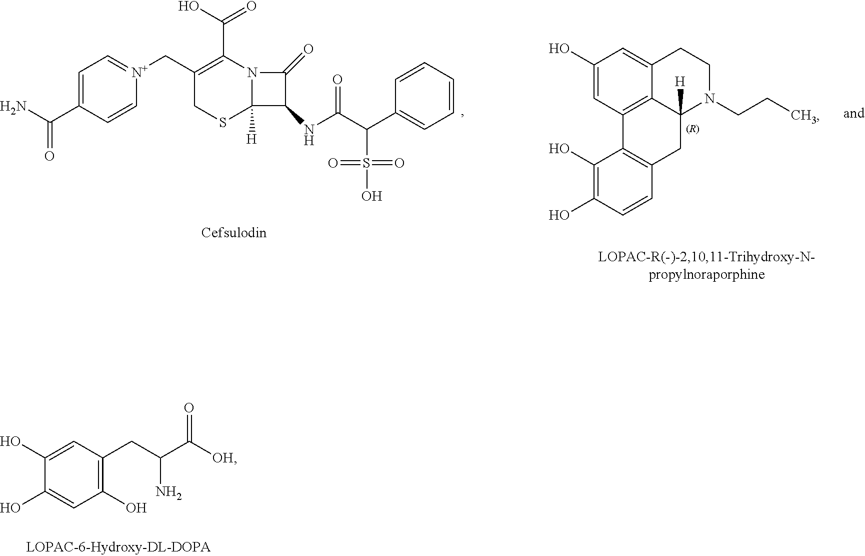

or a pharmaceutically acceptable salt and/or prodrug of any of the foregoing. In certain preferred embodiments, the ENPP1 inhibitor is selected from mesalamine, pentetic acid, methacycline, benserazide, doxylamine, galloflavin, Nitrofurantoin, Chlorpromazine, Disulfiram, Cefotiam, Aurintricarboxylic acid, Myricetin, Bisoprolol, Propantheline bromide, Oxytetracycline, Chicago sky blue 6B, L-3,4-dihydroxyphenylalanine, Meclocycline, Methacholine chloride, LOPAC-SQ 22536, Lymecycline,

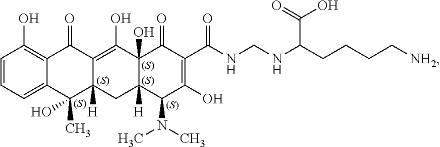

LOPAC-L-DOPS, Cefotaxime, LOPAC-(−)-Epinephrine, Topotecan hydrochloride hydrate, LOPAC-MRS 2159, Pyrocatechol, LOPAC-(−)-alpha-Methylnorepinephrine, Topotecan, PF-477736, Ceftazidime, Minocycline, ARL67156, Bisdemethoxycurcumin,

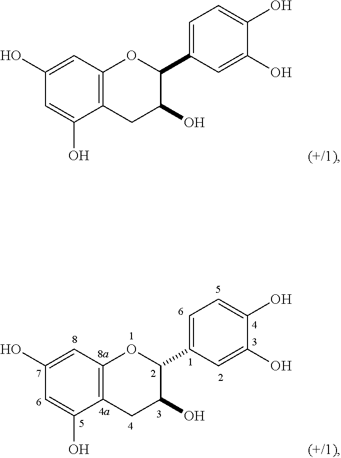 [SMILES: Oc1cc(O)c2c(c1)O[C@@H](c1ccc(O)c(O)c1)[C@@H](O)C2 ; Oc1cc(O)c2c(c1)O[C@H](c1ccc(O)c(O)c1)[C@@H](O)C2]

LOPAC-Ceftriaxone, Cefsulodin, LOPAC-R(−)-2,10,11-Trihydroxy-N-propylnoraporphine, and LOPAC-6-Hydroxy-DL-DOPA, or a pharmaceutically acceptable salt thereof.

In certain embodiments, the ENPP1 inhibitor is selected from mesalamine, oxytetracycline, benserazide, cefotiam, methacycline, cefotaxime, ceftazidime, minocycline, and bisoprolol, or a pharmaceutically acceptable salt and/or prodrug thereof, preferably selected from mesalamine, oxytetracycline, benserazide, cefotiam, methacycline, cefotaxime, ceftazidime, minocycline, and bisoprolol or a pharmaceutically acceptable salt thereof.

In certain embodiments, the ENPP1 inhibitor is ceftazidime or a pharmaceutically acceptable salt and/or prodrug thereof, preferably ceftazidime or a pharmaceutically acceptable salt thereof.

In certain embodiments, the ENPP1 inhibitor is ARL67156 or a pharmaceutically acceptable salt and/or prodrug thereof, preferably ARL67156 or a pharmaceutically acceptable salt thereof.

In certain embodiments, the ENPP1 inhibitor is oxytetracycline or a pharmaceutically acceptable salt and/or prodrug thereof, preferably oxytetracycline or a pharmaceutically acceptable salt thereof.

In certain embodiments, the methods disclosed herein further comprise conjointly administering a bisphosphonate with the ENPP1 inhibitor. Representative bisphosphonates include clondrate, tiludronate, pamidronate, neridronate, olpadronate, alendronate, ibandronate, risedronate, and zoledronate.

In certain aspects, the present disclosure provides methods of inhibiting ATP hydrolysis in ocular tissue, such as Bruch's membrane, comprising contacting the ocular tissue with an ENPP1 inhibitor. In further embodiments, the Bruch's membrane comprises one or more stromal cells.

In certain embodiments, the ENPP1 inhibitor is selected from mesalamine, pentetic acid, methacycline, benserazide, doxylamine, galloflavin, Nitrofurantoin, Chlorpromazine, Disulfiram, Cefotiam, Aurintricarboxylic acid, Myricetin, Bisoprolol, Propantheline bromide, Oxytetracycline, Chicago sky blue 6B, L-3,4-dihydroxyphenylalanine, Meclocycline, Methacholine chloride, LOPAC-SQ 22536, Lymecycline,

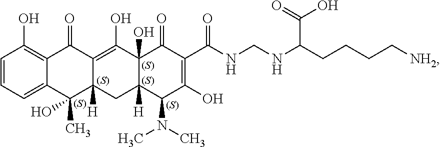

LOPAC-L-DOPS, Cefotaxime, LOPAC-(−)-Epinephrine, Topotecan hydrochloride hydrate, LOPAC-MRS 2159, Pyrocatechol, LOPAC-(−)-alpha-Methylnorepinephrine, Topotecan, PF-477736, Ceftazidime, Minocycline, ARL67156, Bisdemethoxycurcumin,

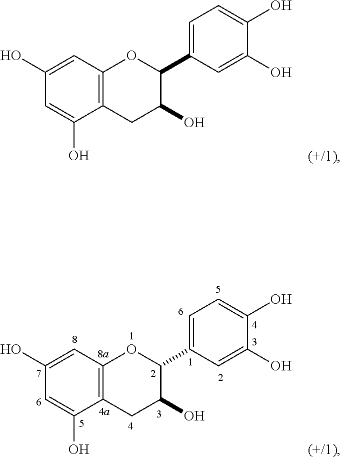 [SMILES: Oc1cc(O)c2c(c1)O[C@@H](c1ccc(O)c(O)c1)[C@@H](O)C2 ; Oc1cc(O)c2c(c1)O[C@H](c1ccc(O)c(O)c1)[C@@H](O)C2]

LOPAC-Ceftriaxone, Cefsulodin, LOPAC-R(−)-2,10,11-Trihydroxy-N-propylnoraporphine, and LOPAC-6-Hydroxy-DL-DOPA, or a pharmaceutically acceptable salt and/or prodrug of any of the foregoing, preferably selected from mesalamine, pentetic acid, methacycline, benserazide, doxylamine, galloflavin, Nitrofurantoin, Chlorpromazine, Disulfiram, Cefotiam, Aurintricarboxylic acid, Myricetin, Bisoprolol, Propantheline bromide, Oxytetracycline, Chicago sky blue 6B, L-3,4-dihydroxyphenylalanine, Meclocycline, Methacholine chloride, LOPAC-SQ 22536, Lymecycline,

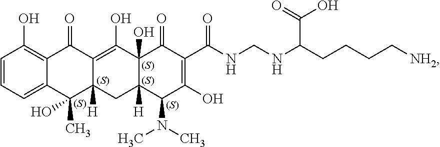

LOPAC-L-DOPS, Cefotaxime, LOPAC-(−)-Epinephrine, Topotecan hydrochloride hydrate, LOPAC-MRS 2159, Pyrocatechol, LOPAC-(−)-alpha-Methylnorepinephrine, Topotecan, PF-477736, Ceftazidime, Minocycline, ARL67156, Bisdemethoxycurcumin,

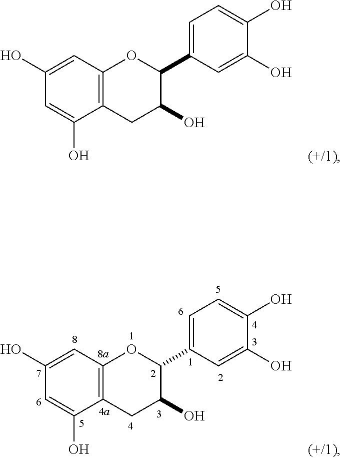 [SMILES: Oc1cc(O)c2c(c1)O[C@@H](c1ccc(O)c(O)c1)[C@@H](O)C2 ; Oc1cc(O)c2c(c1)O[C@H](c1ccc(O)c(O)c1)[C@@H](O)C2]

LOPAC-Ceftriaxone, Cefsulodin, LOPAC-R(−)-2,10,11-Trihydroxy-N-propylnoraporphine, and LOPAC-6-Hydroxy-DL-DOPA, or a pharmaceutically acceptable salt thereof.

In certain embodiments, the ENPP1 inhibitor is selected from mesalamine, oxytetracycline, benserazide, cefotiam, methacycline, cefotaxime, ceftazidime, minocycline, and bisoprolol, or a pharmaceutically acceptable salt and/or prodrug thereof, preferably selected from mesalamine, oxytetracycline, benserazide, cefotiam, methacycline, cefotaxime, ceftazidime, minocycline, and bisoprolol, or a pharmaceutically acceptable salt thereof.

In certain embodiments, the ENPP1 inhibitor is ceftazidime or a pharmaceutically acceptable salt and/or prodrug thereof, preferably ceftazidime, or a pharmaceutically acceptable salt thereof.

In certain embodiments, the ENPP1 inhibitor is ARL67156 or a pharmaceutically acceptable salt and/or prodrug thereof, preferably ARL67156, or a pharmaceutically acceptable salt thereof.

In certain embodiments, the ENPP1 inhibitor is oxytetracycline or a pharmaceutically acceptable salt and/or prodrug thereof, preferably oxytetracycline, or a pharmaceutically acceptable salt thereof.

In certain embodiments, the methods further comprise contacting the ocular tissue with a bisphosphonate, e.g., clondrate, tiludronate, pamidronate, neridronate, olpadronate, alendronate, ibandronate, risedronate, and zoledronate.

In certain embodiments of the methods disclosed herein, the therapeutic may be a prodrug of the ENPP1 inhibitor, e.g., wherein a hydroxyl in the parent compound is presented as an ester or a carbonate, a phosphate or phosphonic acid is presented as an ester or amide derivative, or a carboxylic acid present in the parent compound is presented as an ester. Thus, the prodrug may metabolize to the active parent compound in vivo (e.g., the ester is hydrolyzed to the corresponding hydroxyl, or carboxylic acid).

In certain embodiments, the ocular pathology is pseudoxanthoma elasticum (PXE) or sclero-choroidal calcification. In certain embodiments, the ocular pathology is PXE. In certain embodiments, the ocular pathology is sclero-choroidal calcification.

### Definitions

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the subject of one or more of the disclosed compositions. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “prodrug” is intended to encompass compounds which, under physiologic conditions, are converted into therapeutically active agents. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) and esters or amides of phosphates and phosphonic acids are preferred prodrugs of the present invention.

### Pharmaceutical Compositions

The compositions and methods of the present invention may be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of the disclosed compounds. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, bitartaric, citric, ascorbic, maleic, benzoic, phenylacetic, cinnamic, salicylic, and sulfosalicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds disclosed herein are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds disclosed herein for laboratory use, or for subsequent conversion to a pharmaceutically acceptable acid addition salt.

The term “pharmaceutically acceptable basic addition salt” as used herein means any non-toxic organic or inorganic base addition salt of any acid compounds disclosed herein. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, intraocular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, about 0.1 to about 99.5% (more preferably, about 0.5 to about 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the subject's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of compounds of the invention with one or more additional therapeutic agent(s) provides improved efficacy relative to each individual administration of the compound of the invention or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

This invention includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Example

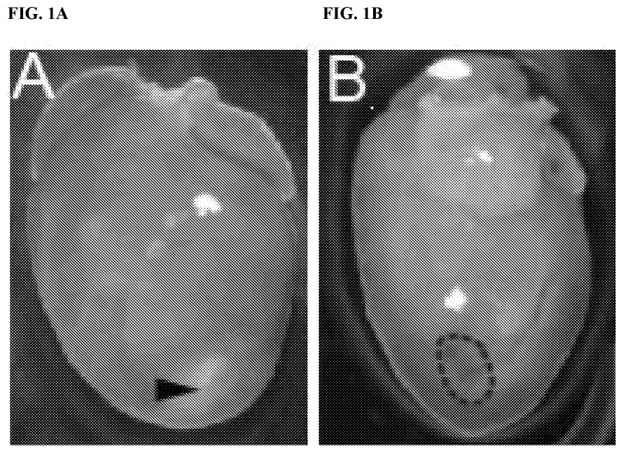

ENPP1 inhibitors attenuate calcification in a mouse model of PXE. Mice genetically deficient in ABCC6 (the causative gene in PXE) were subjected to heart injury, and either (A) vehicle or (B) ARL67156 was administered (intraperitoneally 1 mcg/daily) for 7 days. As shown in FIG. 1, administration of the ENPP1 inhibitor ARL67156 led to significant attenuation of calcification in the injured region (indicated by the arrowhead in FIG. 1A and the circled area in FIG. 1B).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method of treating an ocular pathology characterized by ectopic calcification in a subject, comprising administering to the subject an ENPP1 inhibitor.

2. The method of claim 1, wherein the ocular pathology is pseudoxanthoma elasticum (PXE), sclero-choroidal calcification or choroidocalcinosis.

3. The method of claim 1 or 2, wherein the ocular pathology is pseudoxanthoma elasticum (PXE) or sclero-choroidal calcification.

4. The method of claim 2, wherein the ocular pathology is PXE.

5. The method of claim 2, wherein the ocular pathology is sclero-choroidal calcification.

6. The method of claim 3, wherein the ocular pathology is choroidocalcinosis.

7. The method of any one of claims 1-6, comprising administering the ENPP1 inhibitor ocularly.

8. The method of claim 7, wherein the ocular administration is topically (e.g., as eyedrops).

9. The method of claim 7, wherein the ocular administration is by intraocular injection.

10. The method of claim 7, wherein the ocular administration is via implantation of a device comprising the ENPP1 inhibitor to an eye of the subject.

11. The method of any one of claims 1-10, wherein the subject is an elderly subject.

12. The method of any one of claims 1-10, wherein the subject is a pediatric subject.

13. The method of any one of claims 1-10, wherein the subject is an adult.

14. The method of any one of claims 1-13, wherein the ENPP1 inhibitor is selected from 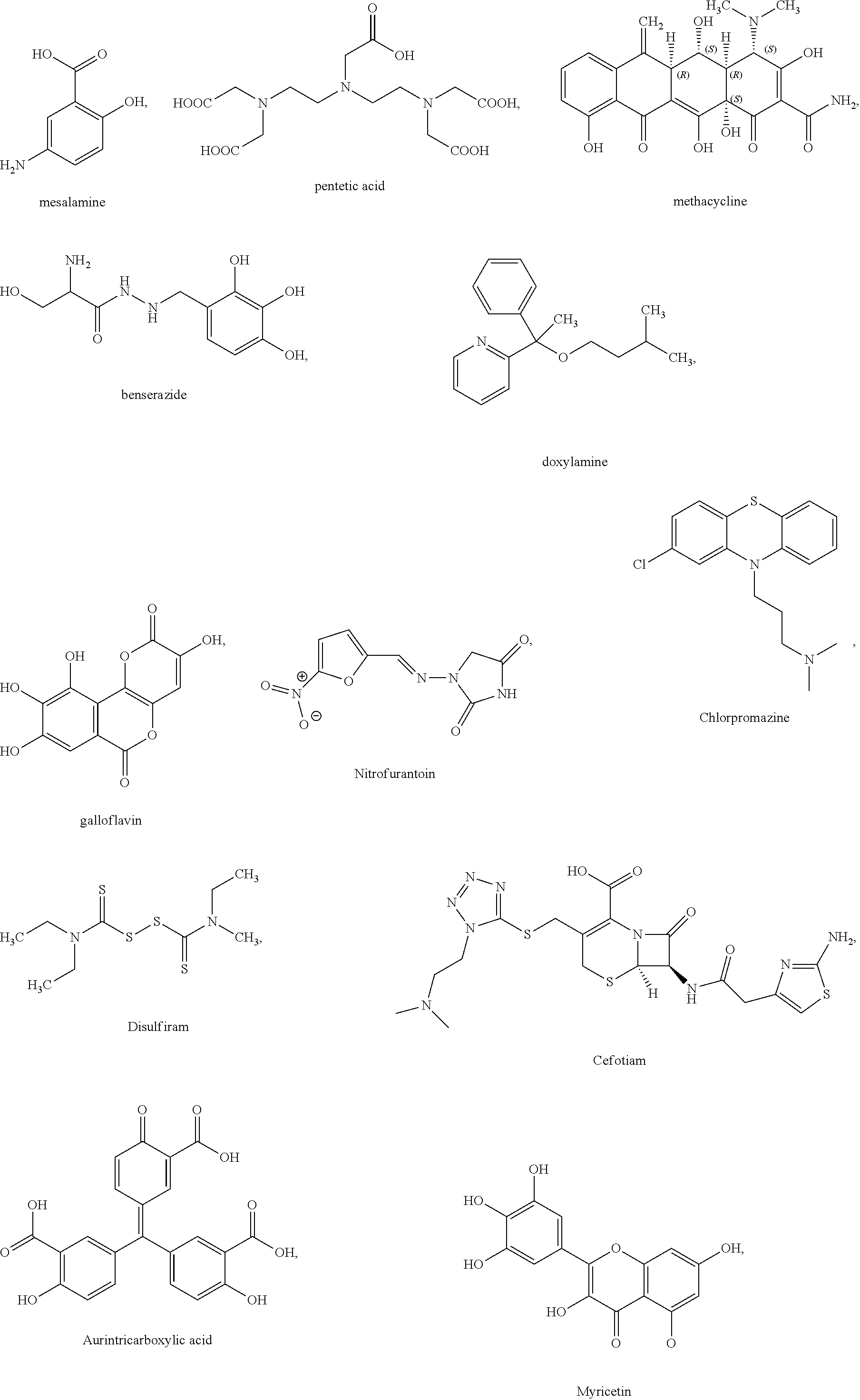 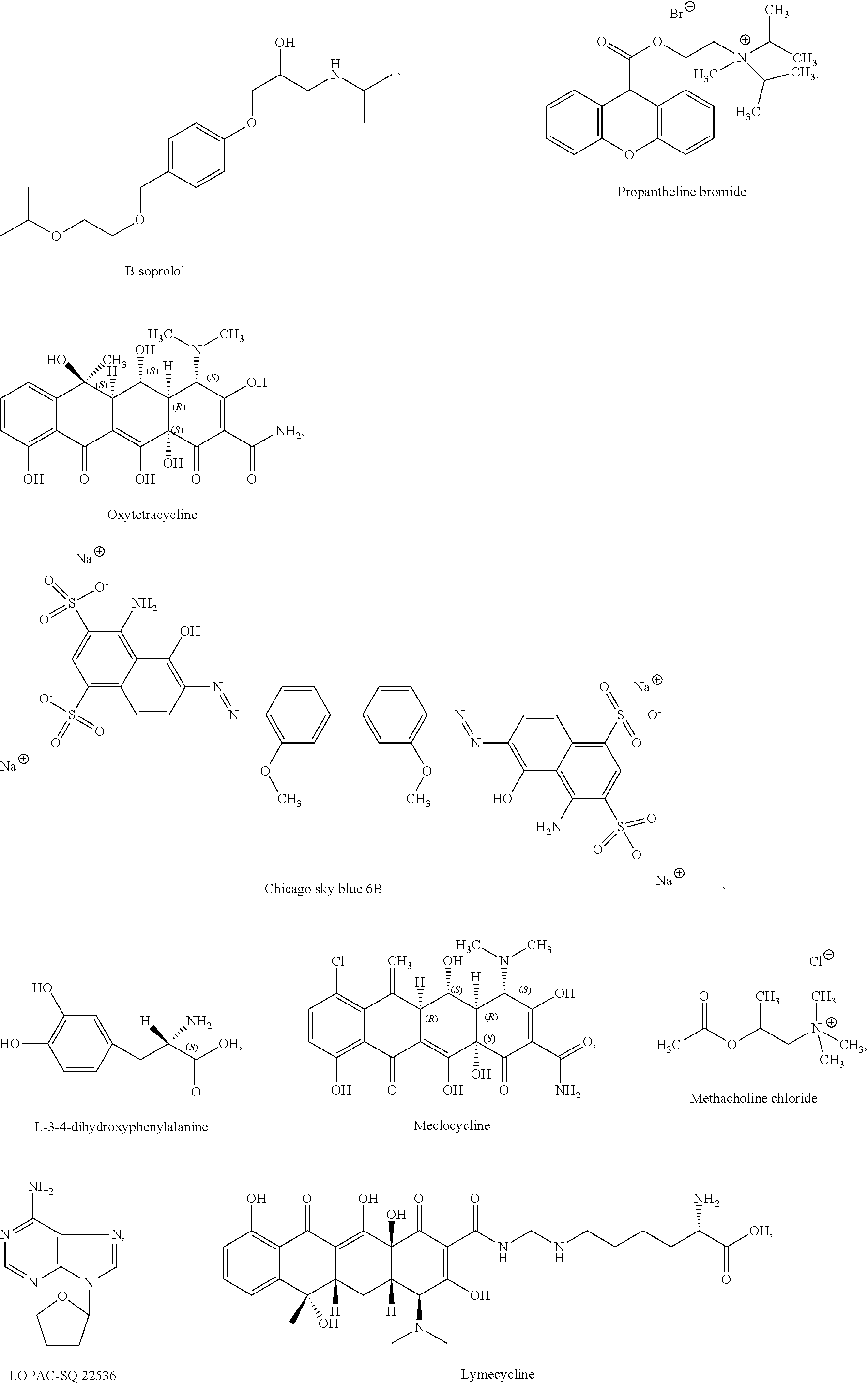 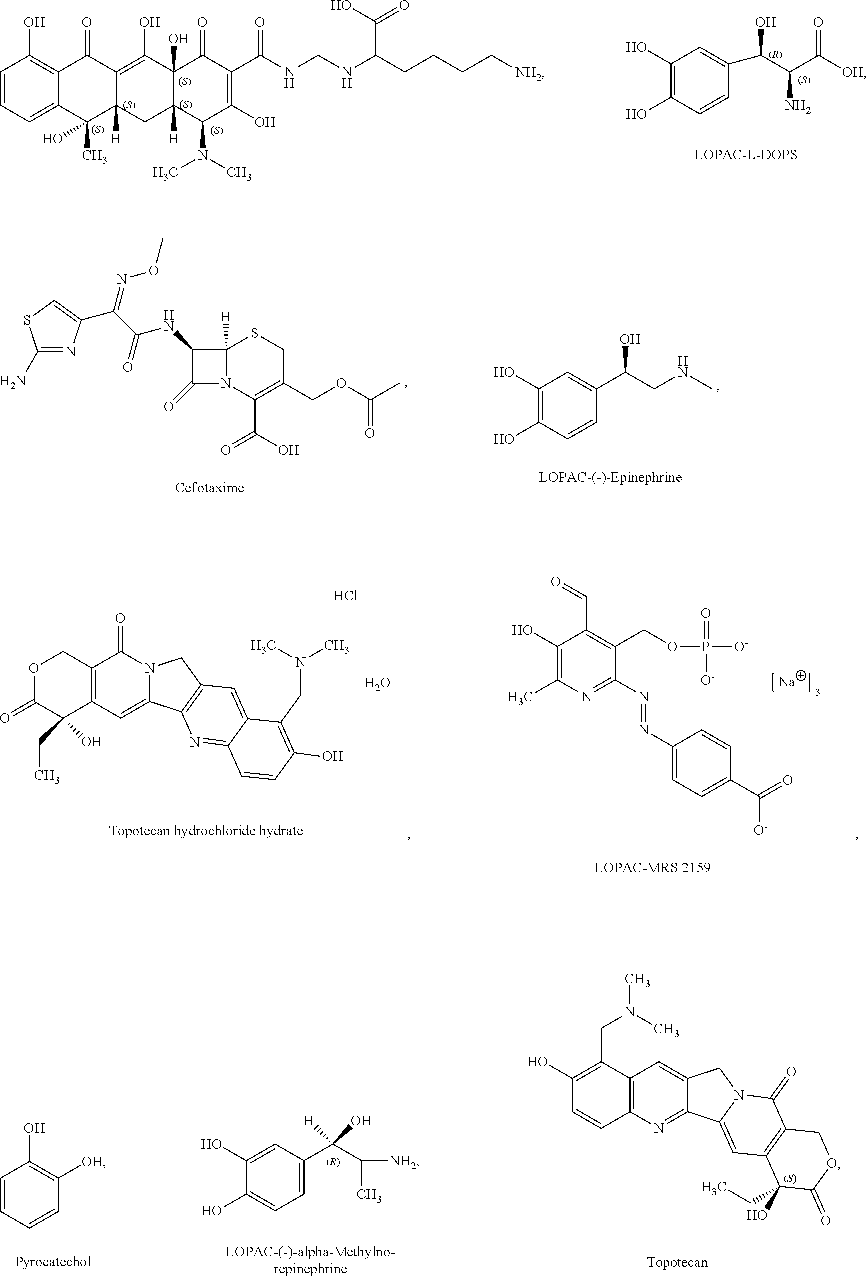 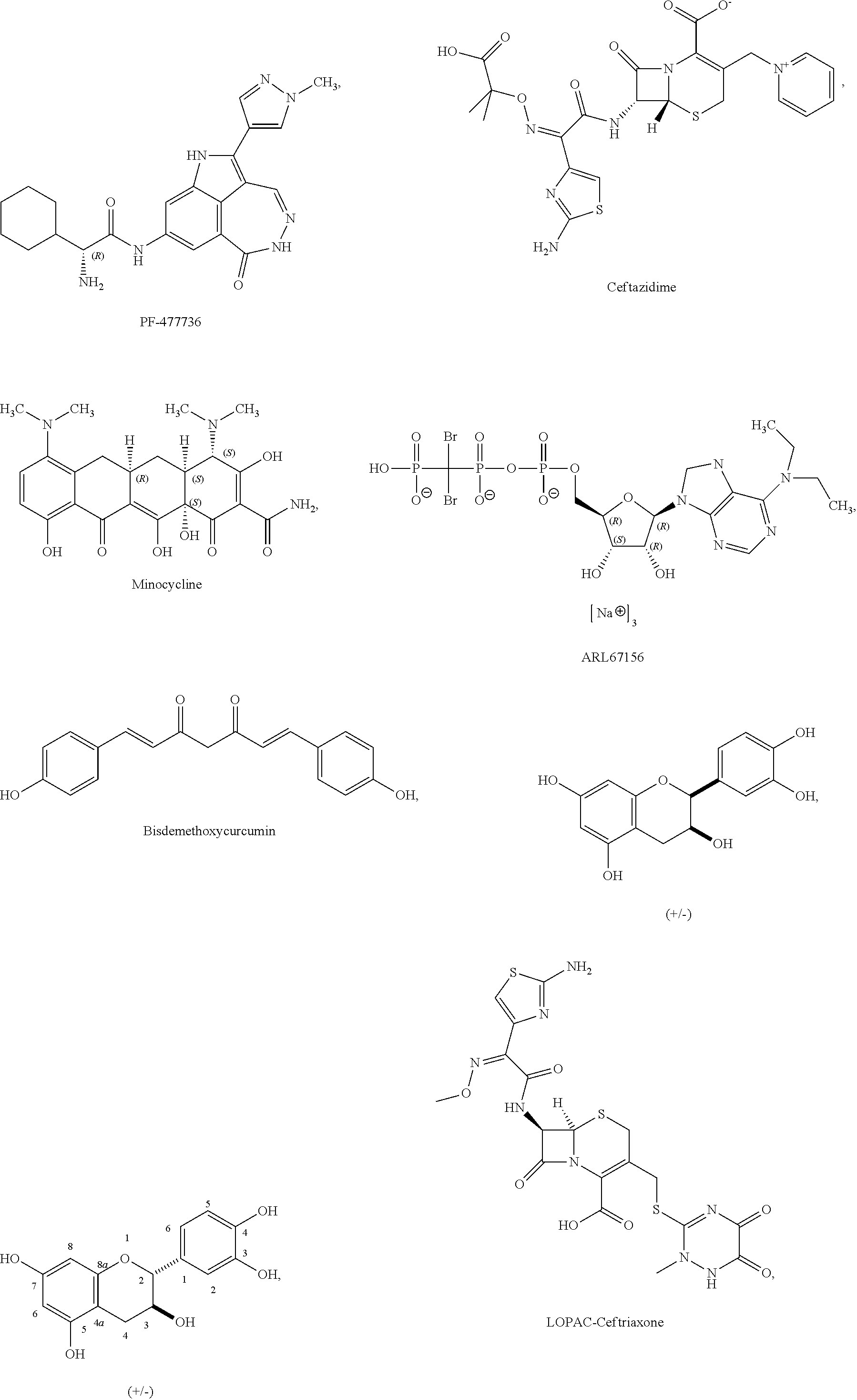 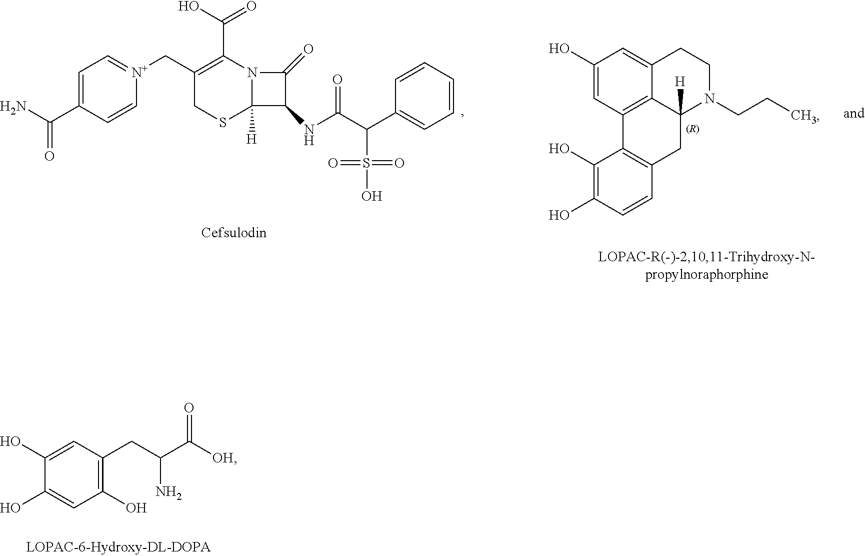
or a pharmaceutically acceptable salt and/or prodrug of any of the foregoing.

15. The method of claim 14, wherein the ENPP1 inhibitor is selected from 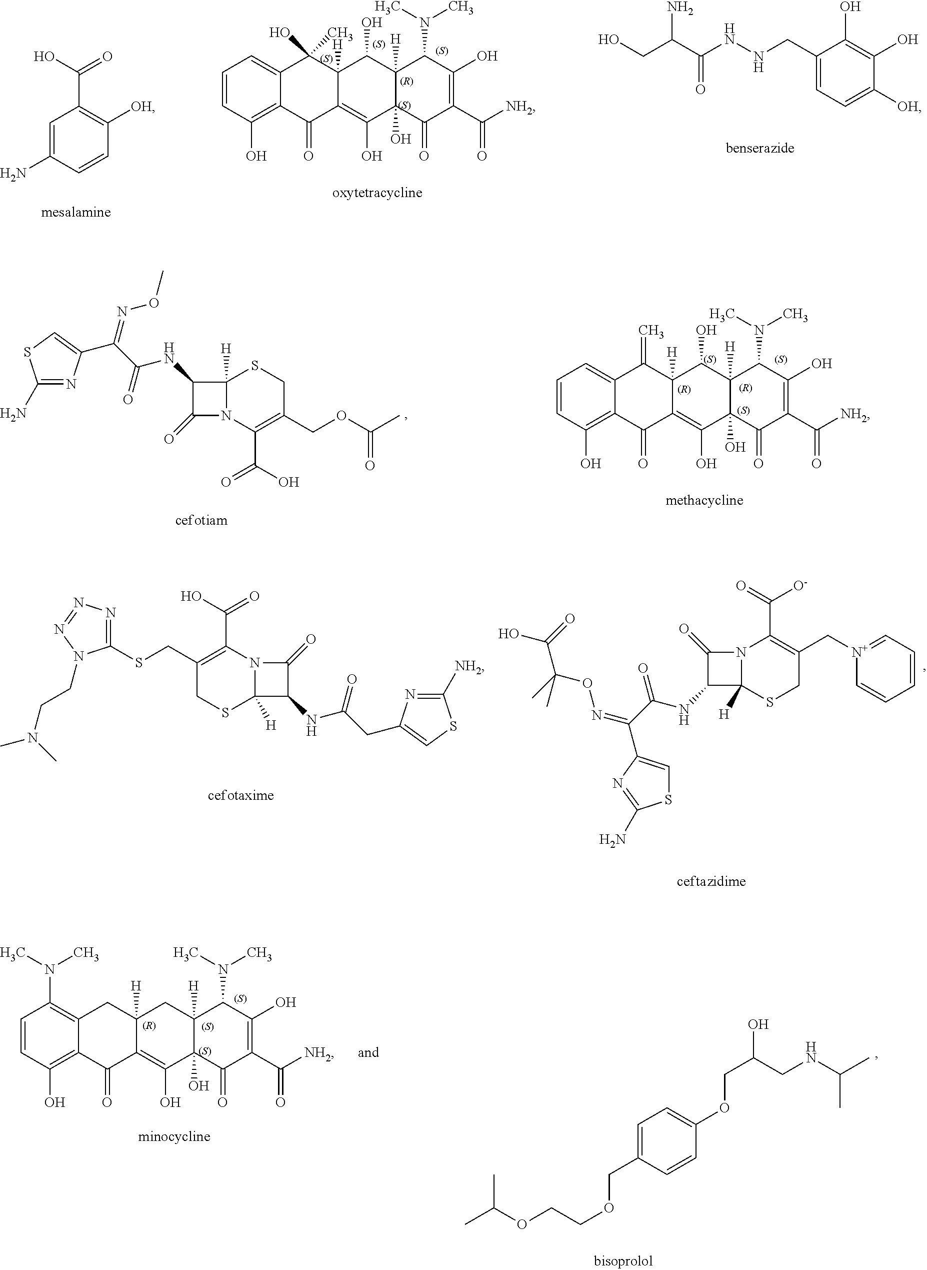
or a pharmaceutically acceptable salt and/or prodrug thereof.

16. The method of any one of claims 1-15, wherein the ENPP1 inhibitor is 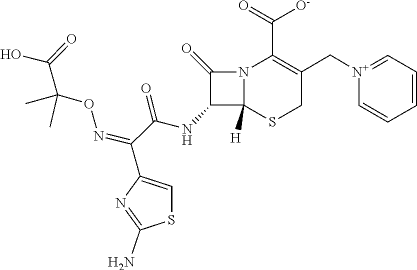 [SMILES: CC(C)(O/N=C(\C(=O)N[C@@H]1C(=O)N2C(C(=O)[O-])=C(C[n+]3ccccc3)CS[C@H]12)c1csc(N)n1)C(=O)O]
ceftazidime or a pharmaceutically acceptable salt and/or prodrug thereof.

17. The method of any one of claims 1-15, wherein the ENPP1 inhibitor is ARL67156 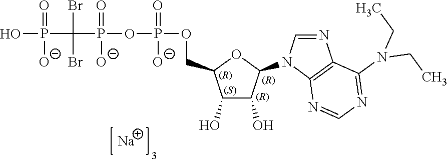
or a pharmaceutically acceptable salt and/or prodrug thereof.

18. The method of any one of claims 1-15, wherein the ENPP1 inhibitor is oxytetracycline 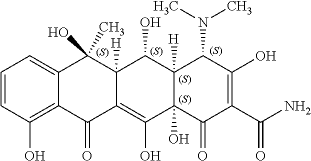
or a pharmaceutically acceptable salt and/or prodrug thereof.

19. The method of any one of claims 1-18, further comprising conjointly administering a bisphosphonate with the ENPP1 inhibitor.

20. The method of claim 19, wherein the bisphosphonate is selected from clondrate, tiludronate, pamidronate, neridronate, olpadronate, alendronate, ibandronate, risedronate, and zoledronate.

21. A method of inhibiting ATP hydrolysis in ocular tissue, comprising contacting the ocular tissue with an ENPP1 inhibitor.

22. The method of claim 21, wherein the ocular tissue comprises Bruch's membrane.

23. The method of claim 22, wherein the Bruch's membrane comprises one or more stromal cells.

24. The method of any one of claims 21-23, wherein the ENPP1 inhibitor is selected from selected from 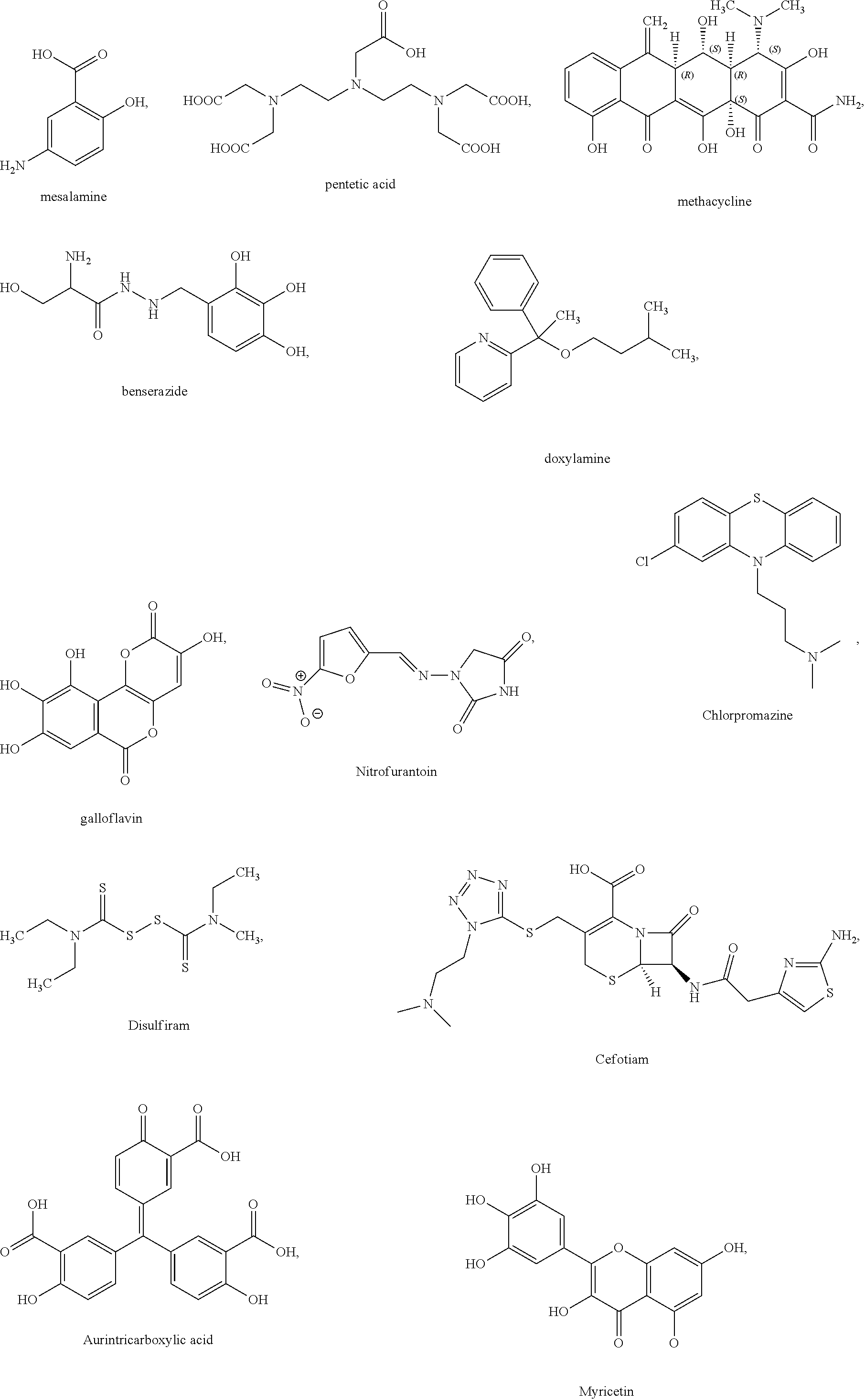 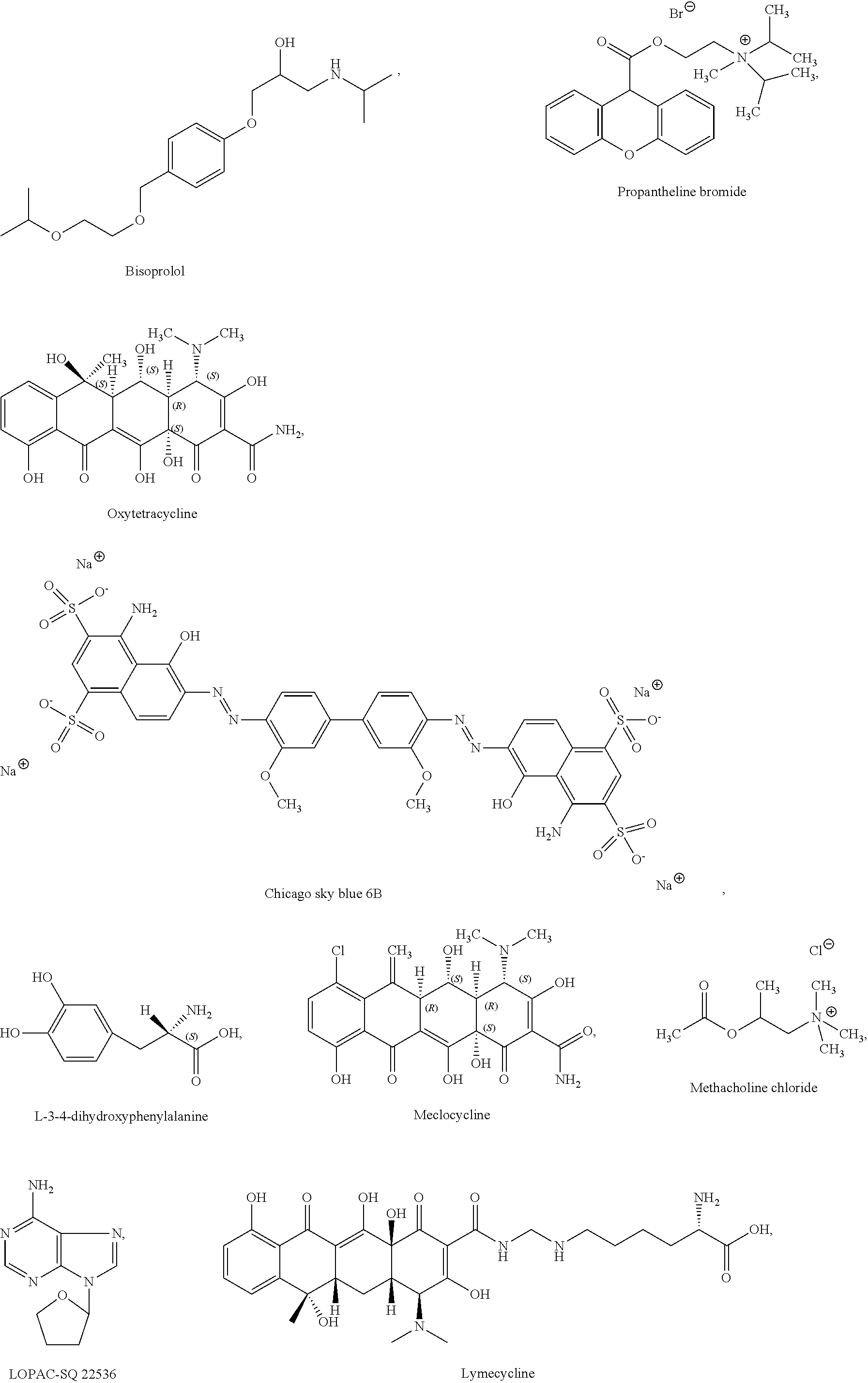 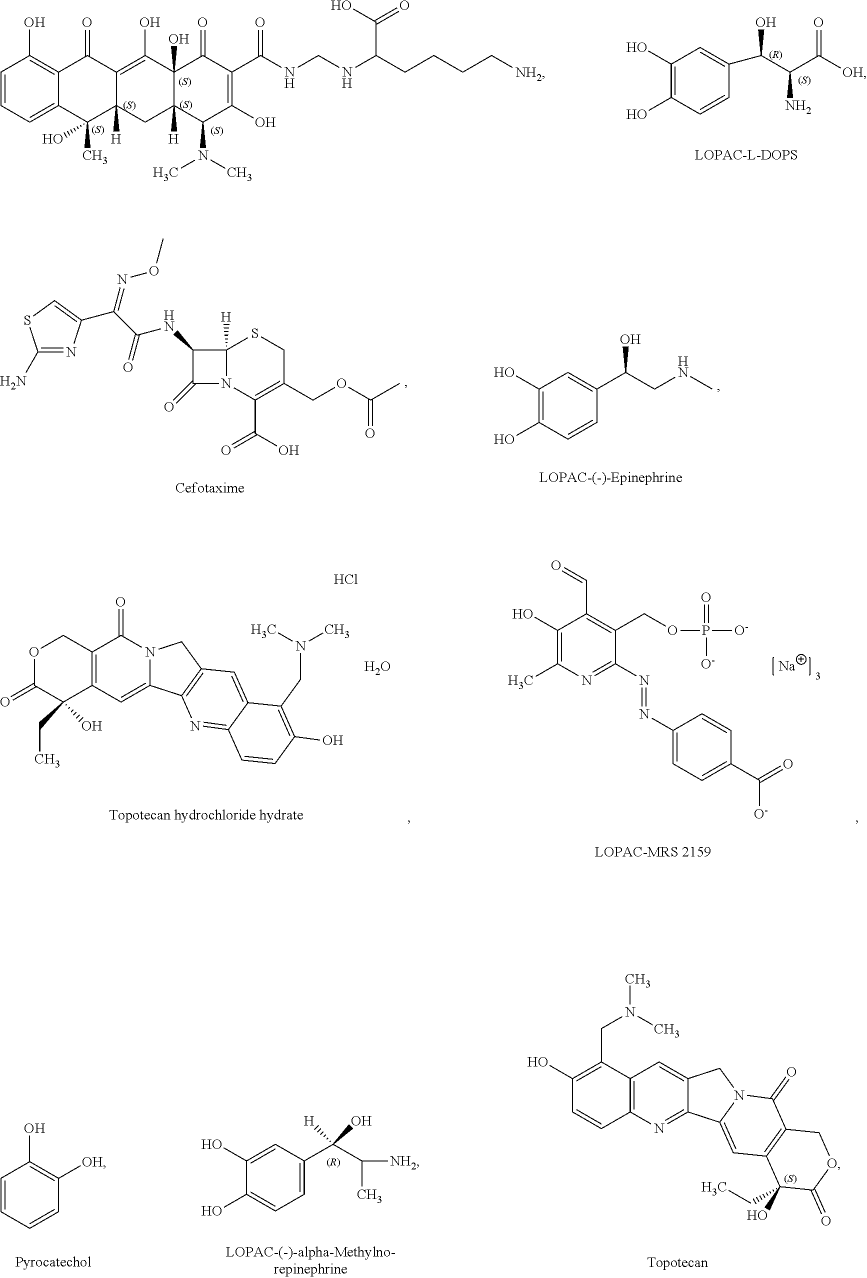 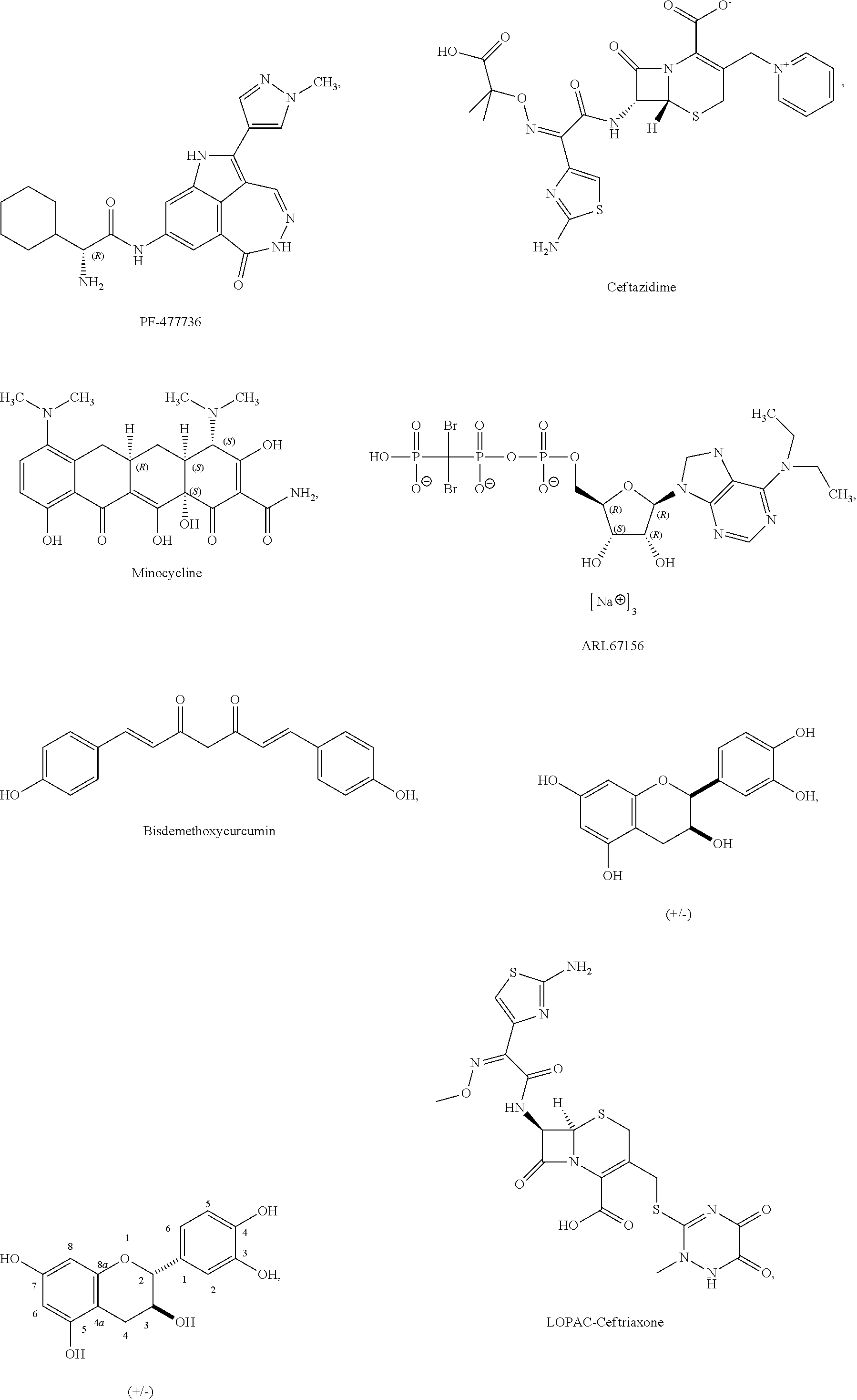 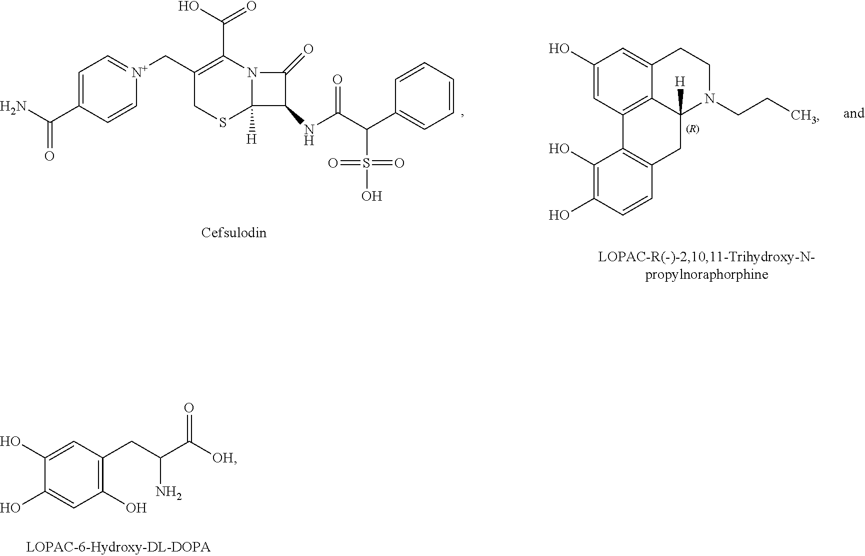
or a pharmaceutically acceptable salt and/or prodrug of any of the foregoing.

25. The method of claim 24, wherein the ENPP1 inhibitor is selected from 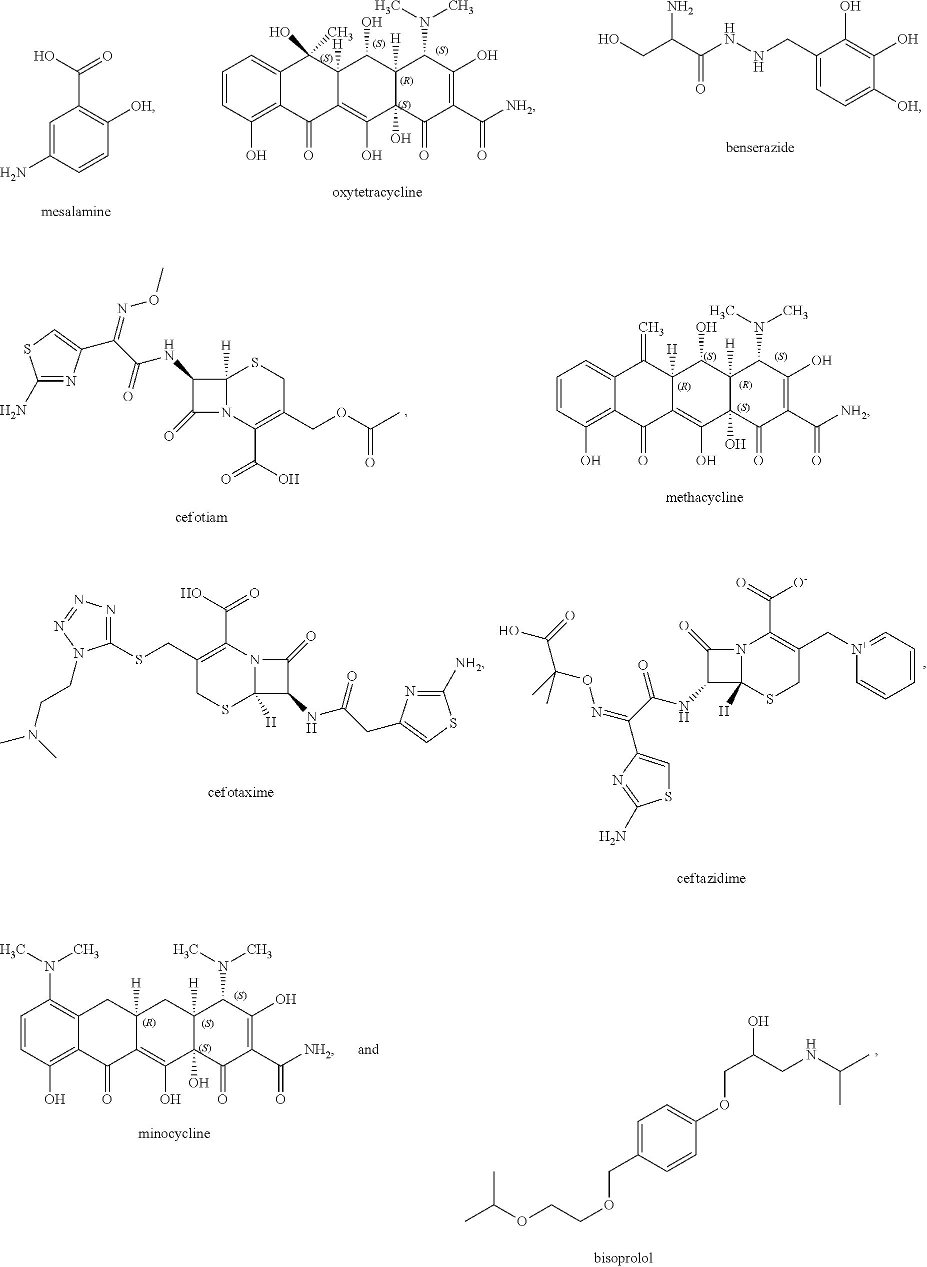
or a pharmaceutically acceptable salt and/or prodrug thereof.

26. The method of any one of claims 21-24, wherein the ENPP1 inhibitor is ceftazidime 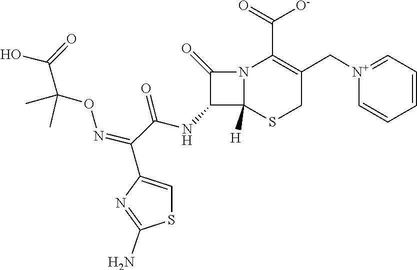 [SMILES: CC(C)(O/N=C(\C(=O)N[C@@H]1C(=O)N2C(C(=O)[O-])=C(C[n+]3ccccc3)CS[C@H]12)c1csc(N)n1)C(=O)O]
or a pharmaceutically acceptable salt and/or prodrug thereof.

27. The method of any one of claims 21-24, wherein the ENPP1 inhibitor is ARL67156 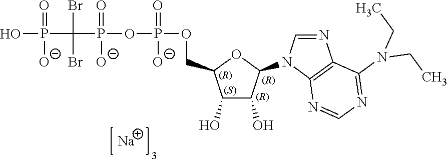
or a pharmaceutically acceptable salt and/or prodrug thereof.

28. The method of any one of claims 21-24, wherein the ENPP1 inhibitor is oxytetracycline 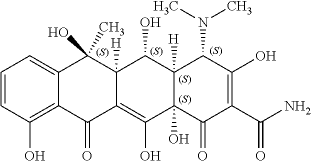
or a pharmaceutically acceptable salt and/or prodrug thereof.

29. The method of any one of claims 21-28, further comprising contacting the ocular tissue with a bisphosphonate.

30. The method of claim 29, wherein the bisphosphonate is selected from clondrate, tiludronate, pamidronate, neridronate, olpadronate, alendronate, ibandronate, risedronate, and zoledronate.
